(19) United States
(12) Patent Application Publication
(10) Pub. No.: US 2025/0262264 A1
(43) Pub. Date: Aug. 21, 2025

(54) FORMULA AND PREPARATION METHOD OF CHINESE HERBAL FEMININE CARE SOLUTION

(71) Applicant: Shuiching WONG, Hong Kong (HK)

(72) Inventors: Shuiching WONG, Hong Kong (HK); Yeungchu CHAN, Hong Kong (HK)

(21) Appl. No.: 19/199,403

(22) Filed: May 6, 2025

Related U.S. Application Data
(63) Continuation of application No. PCT/CN2023/131891, filed on Nov. 15, 2023.

(30) Foreign Application Priority Data
Nov. 17, 2022 (CN) 202211440572.2

(51) Int. Cl. A61K 36/738; A61K 31/045; A61K 33/04; A61K 33/243; A61K 36/534; A61P 31/04

(52) U.S. Cl. CPC A61K 36/738; A61K 31/045; A61K 33/04; A61K 33/243; A61K 36/534; A61P 31/04; A61K 2236/15; A61K 2236/33; A61K 2236/331

(57) ABSTRACT

A formula for a Chinese herbal feminine care solution is made from the following ingredients in parts by weight: 10-15 parts of rose hydrosol, 3-5 parts of peppermint hydrosol, 30-45 parts of a Chinese herbal extract, 3-5 parts of borneol, 1-5 parts of chelated platinum ions, and 6-12 parts of composite antibacterial agent. A preparation method thereof includes: adding the rose hydrosol and the peppermint hydrosol into the Chinese herbal medicine extract to obtain a stock solution; diluting the stock solution; adding the borneol, stirring evenly, and filtering to obtain a filtrate; adding the chelated platinum ions and the composite antibacterial agent into the filtrate to stir evenly, so as to obtain the Chinese herbal feminine care solution. It has excellent bactericidal effects, effectively cleans the external female genitalia, kills bacteria and relieves itching, removes odors, prevents inflammation of the vulva, and has no side effects.

### CROSS-REFERENCE TO RELATED APPLICATIONS

This application is a continuation of International Patent Application No. PCT/CN2023/131891, filed Nov. 15, 2023. The International Patent Application claims priority to a Chinese patent application No. CN202211440572.2 filed on Nov. 17, 2022. The entire contents of the above-mentioned applications are hereby incorporated by reference.

### TECHNICAL FIELD

The disclosure relates to the field of female health care, and more particularly to a formula for a Chinese herbal feminine care solution and a preparation method thereof.

### BACKGROUND

Social responsibilities and life pressures have increased dramatically for modern women, forcing them to cope with a fast-paced lifestyle. They spend most of their time in the office, traveling to and from work, on business trips, meaning they spend much of their time in public places, thereby increasing the likelihood of external infections. According to relevant data, about 85% of adult women in the world suffer from gynecological inflammation to varying degrees, 46% of them have the problem of recurrent disease, and patients show a trend of younger age, 53.9% of students have vaginitis related symptoms, which is mostly bacterial vaginitis (35.9%), followed by candidal vaginitis (23.6%). Therefore, daily antibacterial care is especially important to reduce the occurrence of the gynecological inflammation.

Feminine care solution, also known as vaginal wash or feminine antibacterial wash, is a lotion used by women to clean, eliminate unpleasant odors, and treat the gynecological inflammation, with the aim of killing pathogenic microorganisms in a genital area and disinfection. In fact, men can also use it for disinfection and sterilization of the genital area. As for the existing feminine care solutions on the Chinese market, they can be divided into healthcare types with natural lactic acid and herbal plant essences as main ingredients, and over-the-counter drug types with Chinese herbal medicines as main ingredients.

The existing feminine care solutions typically come in the form of feminine washes or lavage fluid, including cleaning and care types, antibacterial washes, and therapeutic washes. The cleaning and care types are mainly used for daily cleaning the genital area due to normal secretions and excretions, often referred to as care solutions or feminine hygiene washes. The antibacterial washes, as the name suggests, are primarily used for combating and inhibiting bacteria in the feminine intimate area. The therapeutic washes are used when women are infected with various vaginal inflammations, such as Jieeryin wash, Huangku wash, Jiaoyan intimate cleansing wash, etc.

A Chinese Patent No. CN104940122A discloses a no-wash type female care solution and a preparation method thereof. The no-wash type female care solution is obtained by fully dissolving a plant nourishing soft and moist agent, a plant bacteriostasis odor removing agent, a plant anti-allergy antipruritic agent, a microorganism modifier, plant hydrolat, citric acid, sodium citrate and the like into a solvent. Compared with the Chinese Patent No. CN104940122A, the disclosure adds components such as peppermint hydrosol, borneol, chelated platinum ions and a composite antibacterial agent, and uses a specific Chinese herbal formula to prepare a Chinese herbal extract, rather than the traditional Chinese medicine extract of the Chinese Patent No. CN104940122A. Moreover, in the disclosure, a preparation method of the composite antibacterial agent and a dosage ratio of each component are detailed and specified.

A Chinese Patent No. CN113396899A discloses a compound disinfectant of a nano platinum chelate and polyhexamethylene guanidine (PHMG). The product is composed of the nano platinum chelate, the PHMG, a dispersing agent, a stabilizing agent, a complexing agent and water. Compared with the Chinese Patent No. CN113396899A, the composite antibacterial agent of the disclosure adopts a special sulfhydrylated silica (also referred to as thiol-functionalized silica) as a carrier and introduces dodecfluoroheptyl ester and a guanamine group with excellent antibacterial performance into a surface of a nano-carrier, and the composite antibacterial agent is prepared by photopolymerization. Instead of the Chinese Patent No. CN113396899A, the nano platinum chelate and the PHMG are directly mixed and compounded.

A Chinese Patent No. CN108014345A discloses a drug-loaded silica nanoparticle, and a preparation method and an application thereof. The drug-loaded silica nanoparticle contains dimercaptosuccinic acid and a sulfhydrylated mesoporous silica nanoparticle, and the dimercaptosuccinic acid is optionally coupled with the sulfhydrylated mesoporous silica nanoparticle. Compared with the Chinese Patent No. CN108014345A, the disclosure not only prepares the sulfhydrylated silica, but also further prepares the composite antibacterial agent by copolymerizing and introducing dodecfluoroheptyl methacrylate and vinyl guanamine.

A Chinese Patent No. CN101775143A discloses an ultraviolet light-curing compound paint containing fluorine-containing polysiloxane and a preparation method thereof. The fluorine-containing polysiloxane is obtained by mixing hydrogen-containing silicone oil, fluorine-containing (methyl) acrylate and a solvent, and adding allyl glycidyl ether, (methyl) acrylic acid, a catalyst and a polymerization inhibitor for reaction under a protection of nitrogen. Compared with the Chinese Patent No. CN101775143A, the disclosure polymerizes the dodecfluoroheptanyl methacrylate and the vinyl guanamine on the surface of nano-silica to form a special core-shell structure, which is specifically used for the feminine care solution instead of coating.

The above four patents respectively involve related arts, but they belong to different technical fields and there is no clear motivation to combine these technologies. The Chinese Patent No. CN104940122A involves the female care solution with a traditional Chinese medicine formula. The Chinese Patent No. CN113396899A mainly focuses on the application of chelated platinum and guanidine in the medical field after direct compounding. The Chinese Patent No. CN108014345A discusses the application of sulfhydrylated silica in drug delivery, but does not provide inspirations for further improvement based on the sulfhydrylated silica. The Chinese Patent No. CN101775143A only concerns the application of fluoropolymers in the field of coating. These documents do not give any indication or suggestion to combine these discrete technical elements into a specific formula of the disclosure. Those skilled in the art could not foresee the unique effects of the disclosure even assuming that there is a motivation for the combination. In the disclosure, the composite antibacterial agent achieves long-lasting antibacterial effect by introducing the dodecfluoroheptyl ester and the guanamine group with excellent antibacterial performance onto the surface of the nano-silica. The specific Chinese herbal formula works in synergy with other ingredients, providing outstanding anti-inflammatory and soothing effects. The addition of the chelated platinum ions further enhances the antibacterial performance and healing ability of the product.

### SUMMARY

In order to address deficiencies in related art, a purpose of the disclosure is to provide a formula for a Chinese herbal feminine care solution and a preparation method thereof, which has excellent bactericidal effects, can effectively clean external female genitalia (also referred to as vulva), kill bacteria and relieve itching, remove odors, prevent vulvar inflammation, and has no side effects on the human body.

To achieve the above-mentioned purpose, the disclosure adopts technical solutions as follows.

Specifically, a formula for a Chinese herbal feminine care solution is made from raw materials in parts by weight: 10-15 parts of rose hydrosol, 3-5 parts of peppermint hydrosol, 30-45 parts of a Chinese herbal extract, 3-5 parts of borneol, 1-5 parts of chelated platinum ions, and 6-12 parts of composite antibacterial agent.

The formula of the Chinese herbal feminine care solution gives full play to the synergistic effect of the Chinese herbal, and has remarkable antibacterial, anti-inflammatory and anti-itching effects. The rose hydrosol and the peppermint hydrosol can not only clean and soothe the intimate skin, but also bring a fresh and pleasant fragrance. The various herbal components in the Chinese herbal extract are mutually compatible, comprehensively enhancing the anti-inflammatory and antibacterial effects of the product. The borneol incense has antibacterial, analgesic and anti-inflammatory effects, and can effectively relieve uncomfortable symptoms. The chelated platinum ions can promote skin repair and regeneration. The overall formula not only effectively prevents and alleviates various gynecological inflammations, but also maintains the pH balance of the vagina, improves the micro-ecological environment of the intimate area, and provides comprehensive protection for women's daily care.

A preparation method of the composite antibacterial agent includes the following steps.

Preparation of sulfhydrylated silica: this step is a surface modification process aimed at introducing a sulfhydryl group (—SH) onto a surface of silica. The specific process is as follows. Nano-silica is dispersed in an ethanol aqueous solution, ammonia water and 3-mercaptopropyltrimethoxysilane are added for reaction through stirring, so as to obtain a reaction product (i.e., first product). Finally, the reaction product is centrifuged, washed and dried sequentially to obtain the sulfhydrylated silica. The reaction mechanism is as follows: the ammonia water acts as a catalyst to promote the hydrolysis of 3-mercaptopropyltrimethoxysilane, generating a silanol intermediate. The silanol intermediate undergoes condensation reaction with a hydroxyl group (-OH) on the surface of silica to form a Si—O—Si bond and simultaneously release a water molecule. After the reaction is completed, the surface of silica is modified by the sulfhydryl group, forming the sulfhydrylated silica.
(1) The nano-silica is dispersed into the ethanol aqueous solution to obtain a first dispersed solution, then the ammonia water and the 3-mercaptopropyltrimethoxysilane are added into the first dispersed solution to obtain a first mixed solution, the first mixed solution is stirred for a reaction to obtain the first product, and the first product is centrifuged, washed and dried sequentially to obtain the sulfhydrylated silica.

Preparation of the composite antibacterial agent: this step is a photoinitiated free radical polymerization reaction, with the aim of grafting functional monomers onto the surface of the sulfhydrylated silica. The specific process is as follows. Dodecfluoroheptyl methacrylate and vinyl guanamine are dissoved into N,N-dimethylformamide to a mixed solution (i.e., second mixed solution), sulfhydrylated silica is dispersed into the mixed solution by ultrasonic dispersion to obtain a dispersed solution (i.e., second dispersed solution), a photoinitiator is added into the dispersed solution for a reaction under an irradiation of an ultraviolet light to obtain a product (i.e., second product). Finally, the product is centrifuged, washed and dried sequentially to obtain the composite antibacterial agent. The reaction mechanism is as follows. Under the irradiation of the ultraviolet light, the photoinitiator decomposes to generate free radicals, which open double bonds of the dodecfluoroheptyl methacrylate and the vinyl guanamine, forming active monomer free radicals. These free radicals undergo addition reactions with the sulfhydryl group on the surface of sulfhydrylated silica, grafting functional monomers onto the surface of silica. The polymerization reaction proceeds continuously, eventually forming a composite structure with a hydrophobic antibacterial group (from dodecfluoroheptyl methacrylate) and a hydrophilic antibacterial group (from vinyl guanamine).
(2) The dodecfluoroheptyl methacrylate and the vinyl guanamine are added to the N,N-dimethylformamide, stirred evenly to obtain the second mixed solution. Then, the sulfhydrylated silica is dispersed into the second mixed solution by ultrasonic dispersion to obtain the second dispersed solution. The photoinitiator is added into the second dispersed solution for the reaction under the irradiation of the ultraviolet light to obtain the second product. The second product is centrifuged, washed with isopropanol, and dried to obtain the complex antibacterial agent.

In an embodiment, in the step (1), a concentration of the ethanol aqueous solution is in a range of 50 weight percent (wt %) to 75wt %, and a concentration of the ammonia water is in a range of 15wt % to 25wt %.

In an embodiment, in the step (1), a weight ratio of the nano-silica, the ethanol aqueous solution, the ammonia water and the 3-mercaptopropyltrimethoxylsilane is 10: (50-70): (8-17): (3-6).

In an embodiment, in the step (1), conditions of the reaction include: 5 to 8 hours (h) of reaction tine, 20 to 35 Celsius degrees (C.) of reaction temperature, and a nitrogen (N2) atmosphere.

In an embodiment, in the step (2), a weight ratio of the dodecfluoroheptadyl methacrylate, the vinyl guanamine, the N,N-dimethylformamide and the sulfhydrylated silica is (2-5): (4-7): (30-50): 10.

In an embodiment, in the step (2), the photoinitiator is one or more selected from the group consisting of consisting of 2,2-dimethoxy-2-phenylacetophenone, 2-ethoxy-1,2-diphenylethanone, and 2-butoxy-1,2-diphenylethanone.

In an embodiment, in the step (2), time of the ultrasonic dispersion is in a range of 5 to 15 minutes (min), a main wavelength of the ultraviolet light is in a range of 360 to 365 nanometers (nm), an intensity of the ultraviolet light is in a range of 50 to 100 milliwatts per square centimeter (mW/cm²), time of the irradiation is in a range of 30 to 60 min, and a temperature of the reaction is in a range of 30 to 40° C.

The composite antibacterial agent has a unique preparation method and structure. Firstly, by introducing the sulfhydryl group onto the surface of the nano-silica, the dispersibility of the nano-silica is improved. Secondly, the introduction of the dodecfluoroheptyl methacrylate endows the material with hydrophobicity, which is conducive to the dispersion and stability of the antibacterial agent in the care solution. Secondly, the introduction of the vinyl guanamine enhances the hydrophilicity and antibacterial performance of the material, enabling the antibacterial agent to interact better with the bacterial cell wall. Finally, the composite antibacterial agent has a broad-spectrum antibacterial effect, has a good inhibitory effect on a variety of pathogenic bacteria, and is not prone to drug resistance. The application of the composite antibacterial agent has significantly enhanced the antibacterial effect and durability of the Chinese herbal feminine care solution of the disclosure, providing users with a safer and more effective option for intimate area care.

In an embodiment, a preparation method of the Chinese herbal extract includes the following steps: weighing, in parts by weight, 1 to 2 parts of safflower, 8 to 12 parts of peach seed, 8 to 12 parts of Forsythia suspensa, 8 to 13 parts of chrysanthemum, 7 to 13 parts of Perilla frutescens, 18 to 25 parts of Platycodon grandiflorus, 8 to 11 parts of star anise, 9 to 14 parts of Aucklandia lappa, 7 to 12 parts of Agastache rugosa, 10 to 12 parts of Atractylodes lancea, 10 to 12 parts of Atractylodes macrocephala, 9 to 13 parts of green tangerine peel, 10 to 12 parts of cloves, 4 to 6 parts of Zanthoxylum bungeanum, and 13 to 16 parts of tea leaves to obtain a Chinese herbal mixture, adding the Chinese herbal mixture into a reaction vessel, then adding 500 parts of deionized water into the reaction vessel to obtain a to-be-extracted Chinese herbal mixture, and performing distillation extraction on the to-be-extracted Chinese herbal mixture at a temperature in a range of 94-97° C. for 1.5-3 h to obtain the Chinese herbal extract.

The disclosure also provides a preparation method of a Chinese herbal feminine care solution, including the following steps: adding the rose hydrosol and the peppermint hydrosol into the Chinese herbal medicine extract to obtain a stock solution; diluting the stock solution to obtain a diluted stock solution; adding the borneol, stirring the diluted stock solution with the borneol to obtain a stirred solution, and filtering the stirred solution to obtain a filtrate, adding the chelated platinum ions and the composite antibacterial agent into the filtrate to stir evenly, so as to obtain the Chinese herbal feminine care solution.

In an embodiment, a dilution factor of the stock solution is in a range of 10-20 times.

Compared with the related art, the disclosure has the following beneficial effects.

The safflowerhas effects of activating blood circulation and unblocking meridians; dissipating blood stasis and relieving pain. The safflower is used to treat amenorrhea, dysmenorrhea, lochiorrhea, chest impediment and angina, stagnant abdominal pain, hypochondriac stabbing pain, bruises and pain from sores.

The peach seed has effects of activating blood circulation and resolving stasis, moistening the intestines to relieve constipation, and suppressing cough and alleviating asthma. The peach seed is used to treat amenorrhea and dysmenorrhea, abdominal masses and stagnant lumps, pulmonary abscess and intestinal abscess, bruises, intestinal dryness and constipation, and cough and asthma.

The Forsythia suspensa has effects of clearing heat, detoxifying, dissipating nodules, and reducing swelling. The Forsythia suspensa is used to treat warm pathogen syndrome, erysipelas, skin rashes, carbuncles, scrofula, and dysuria.

The chrysanthemum has effects of dispersing wind-heat, clearing heat, brightening eyes and detoxifying. The chrysanthemum is used to treat headache, dizziness, red eyes, restlessness in the chest and heart, boils, and abscesses.

The Perilla frutescens: leaves are diaphoretic, antitussive, and aromatic stomachic diuretics, have effects of analgesic, sedative, and detoxifying, and are used to treat colds; stems can regulate Qi and prevent miscarriage; seeds can suppress cough, eliminate phlegm, alleviate asthma, and relieve mental depression.

The Platycodon grandiflorus has effects of diffusing lung Qi, soothing throat, eliminating phlegm and draining pus. The Platycodon grandiflorus is used to treat excessive phlegm in cough, chest tightness and discomfort, sore throat, hoarseness, expectoration of pus in lung abscess, and non-ulceration of pus in sores and ulcers.

The star anise is warm in nature and pungent in taste, has effects of warming yang to dispel cold and regulating Qi to relieve pain, and is used to treat cold-induced vomiting and hiccups, abdominal pain caused by cold hernia, kidney deficiency and lower back pain, and and dry or damp beriberi.

The Aucklandia lappa has effects of promoting Qi circulation to relieve pain and strengthening the spleen to aid digestion. The Aucklandia lappa is used to treat chest and epigastric distension and pain, tenesmus in diarrhea and dysentery, indigestion due to food retention, and loss of appetite.

The Agastache rugosa has effects of promoting Qi circulation, harmonizing the middle energizer, dispelling foulness, and removing dampness. The Agastache rugosa is used to treat summer-damp colds, chills and fever, headache, stuffiness in chest and epigastrium, vomiting and diarrhea, malaria, dysentery, and halitosis.

The Atractylodes lancea has effects of strengthening the spleen, drying dampness, resolving depression, and dispelling foulness. The Atractylodes lancea is used to treat spleen dysfunction caused by excessive dampness, fatigue and drowsiness, epigastric fullness and abdominal distension, loss of appetite, vomiting, diarrhea, dysentery, malaria, phlegm retention, edema, seasonal colds, wind-cold-damp arthralgia, foot weakness, and night blindness.

The Atractylodes macrocephala has effects of tonifying the spleen, benefiting the stomach, drying dampness, harmonizing the middle energizer, and preventing miscarriage. The Atractylodes macrocephala is used to treat weak spleen and stomach Qi, loss of appetite, fatigue with shortness of breath, abdominal distension, diarrhea, phlegm retention, edema, jaundice, dampness-induced arthralgia, difficulty in urination, dizziness, spontaneous sweating, and unstable fetal condition.

The green tangerine peel has effects of soothing the liver and breaking Qi stagnation, and eliminating food accumulation and resolving stagnation. The green tangerine is used to treat distending pain in the chest and hypochondrium, hernia, breast nodules, mastitis, and abdominal pain due to food accumulation.

The cloves have effects of warming the middle energizer, tonifying the kidneys, and descending rebellious Qi. The cloves are used to treat hiccups, vomiting, regurgitation, diarrhea and dysentery, cold pain in the heart and abdomen, abdominal masses, hernia, and skin diseases.

The Zanthoxylum bungeanum has effects of warming the middle energizer and dispelling cold, removing dampness, relieving pain, killing parasites, and detoxifying fishy smells. The Sichuan pepper is used to treat food retention and fluid retention, cold pain in the heart and abdomen, vomiting, hiccups, cough with reversed Qi flow, arthralgia caused by wind-cold-dampness, diarrhea, dysentery, hernia pain, toothache, ascariasis, enterobiasis, pruritus vulvae, and scabies.

The tea leaves have effects of clearing the head and eyes, relieving irritability and thirst, resolving phlegm, aiding digestion, promoting diuresis, and detoxifying. The tea leaves are used to treat headache, blurred vision, excessive sleepiness, restlessness and thirst, food retention and phlegm stagnation, malaria, and dysentery.

The borneol is used for treating loss of consciousness in closed syndrome, red and swollen eyes, sore throat and mouth ulcers, abscesses and sores with swelling pain that do not heal after rupture, angina pectoris, etc.
1) The disclosure discloses the Chinese herbal feminine care solution, which is formulated and synthesized from the plant components with anti-inflammatory, antioxidant, anti-allergy, infection reduction and immune enhancement functions, the compound antibacterial components, and the chelated platinum ions that play a catalytic role in improving the performance of herbal. The Chinese herbal feminine care solution can effectively clean the female vulva, providing nourishment, odor removal, itch relief, allergy prevention, and antibacterial effects. It also effectively moisturizes and cares for the skin in the female intimate area. It can be used spraying, eliminating the need for traditional washing or douching methods, making it convenient and quick.
2) The disclosure provides the composite antibacterial agent. Firstly, the nano-silica is organically modified with the 3-mercaptopropyltrimethoxysilane to obtain the sulfhydrylated silica, which can effectively improve the dispersibility of the nano-silica and prevent its aggregation and sedimentation in the care solution. Subsequently, under ultraviolet light irradiation, through the sulfhydryl-ene reaction, the dodecfluoroheptyl methacrylate and the vinyl guanamine are grafted onto the sulfhydrylated silica to obtain the composite antibacterial agent. The vinyl guanamine carries a positive charge in water and can adsorb on the surface of negatively charged microorganisms, penetrate into the microorganisms, and disrupt the normal metabolic process of microorganisms, rendering bacteria and viruses inactive. Moreover, it has almost no toxicity to normal eukaryotic cells, is safe and non-toxic to the human body, and does not induce bacterial resistance. Therefore, it can provide excellent antimicrobial activity. The dodecfluoroheptadyl methacrylate has excellent hydrophobic properties, which can prevent the initial adhesion of bacteria and proteins, inhibit the formation of biofilms, and thus work in synergy with the vinyl guanamine to inhibit and kill bacteria.

### DETAILED DESCRIPTION OF EMBODIMENTS

To make the objectives, technical solutions and advantages of the disclosure clearer, the disclosure will be further described in detail with reference to embodiments. It should be noted that, the specific embodiments described here are only used to explain the disclosure, and are not used to limit the disclosure.

Unless otherwise indicated, chemical reagents and materials in the disclosure are purchased by market means or synthesized from raw materials purchased by market means.

A particle size of nano-silica is in a range of 75 to 120 nm.

Both rose hydrosol and peppermint hydrosol are purchased from Guangzhou Zhonghe International Biotechnology Co., Ltd.

Chelated platinum ions are purchased from Yatian Technology Group.

The disclosure will be further described with reference to specific embodiments.

### Embodiment 1

A preparation method of a Chinese herbal feminine care solution includes steps as follows.
(1) 10 grams (g) of nano-silica is dispersed into 70 g of 75wt % ethanol aqueous solution to obtain a first dispersed solution, then 17 g of 20wt % ammonia water and 6 g of 3-mercaptopropyltrimethoxysilane are added into the first dispersed solution to obtain a first mixed solution. The first mixed solution is stirred and reacted in a N₂ atmosphere at 35° C. for 8 h to obtain a first product. The first product is centrifuged, washed and dried to obtain sulfhydrylated silica.
(2) 5 g of dodecfluoroheptyl methacrylate and 7 g of vinyl guanamine are added into 50 g of N,N-dimethylformamide, stirred evenly to obtain a second mixed solution, then 10 g of sulfhydrylated silica is dispersed into the second mixed solution by ultrasonic dispersion for 15 min to obtain a second dispersed solution, and 0.2 g of 2-ethoxy-1,2-diphenylethanone is added in the second dispersed solution. Under an irradiation of ultraviolet light with a main wavelength of 365 nm and an intensity of 100 mW/cm², a reaction is carried out at 40° C. for 60 min to obtain a second product. The second product is centrifuged, washed with isopropanol, dried, so as to obtain the composite antibacterial agent.
(3) 2 g of safflower, 10 g of peach seed, 10 g of Forsythia suspensa, 11 g of chrysanthemum, 10 g of Perilla frutescens, 22 g of Platycodon grandiflorus, 9 g of star anise, 12 g of Aucklandia lappa, 8 g of Agastache rugosa, 11 g of Atractylodes lancea, 11 g of Atractylodes macrocephala, 11 g of green tangerine peel, 11 g of cloves, 5 g of Zanthoxylum bungeanum, and 15 g of tea leaves to obtain a Chinese herbal mixture, and the Chinese herbal mixture is added into a reaction vessel. Then, 500 g of deionized water is added into the reaction vessel to obtain a to-be-extracted Chinese herbal mixture. The to-be-extracted Chinese herbal mixture is performed with distillation extraction at 96° C. for 2.5 h to obtain the Chinese herbal extract.
(4) 15 g of rose hydrosol and 5 g of peppermint hydrosol are added into 45 g of Chinese herbal extract to obtain a stock solution. The stock solution is diluted 20 times to obtain a diluted stock solution, then 5 g of borneol is added into the diluted stock solution, stirred thoroughly and filtered to obtain a filtrate. 5 g of chelating platinum ions and 12 g of composite antibacterial agent are added into the filtrate, stirred evenly, so as to obtain the Chinese herbal feminine care solution.

### Embodiment 2

A preparation method of a Chinese herbal feminine care solution includes steps as follows.
(1) 10 g of nano-silica is dispersed into 60 g of 75wt % ethanol aqueous solution to obtain

a first dispersed solution, then 12 g of 20wt % ammonia water and 5 g of 3-mercaptopropyltrimethoxysilane are added into the first dispersed solution to obtain a first mixed solution. The first mixed solution is stirred and reacted in a N₂ atmosphere at 30° C. for 6 h to obtain a first product. The first product is centrifuged, washed and dried to obtain sulfhydrylated silica.
(2) 4 g of dodecfluoroheptyl methacrylate and 5 g of vinyl guanamine are added into 40 g of N,N-dimethylformamide, stirred evenly to obtain a second mixed solution, then 10 g of sulfhydrylated silica is dispersed into the second mixed solution by ultrasonic dispersion for 10 min to obtain a second dispersed solution, and 0.2 g of 2-ethoxy-1,2-diphenylethanone is added in the second dispersed solution. Under an irradiation of ultraviolet light with a main wavelength of 365 nm and an intensity of 100 mW/cm², a reaction is carried out at 35° C. for 50 min to obtain a second product. The second product is centrifuged, washed with isopropanol, dried, so as to obtain the composite antibacterial agent.
(3) 2 g of safflower, 10 g of peach seed, 10 g of Forsythia suspensa, 11 g of chrysanthemum, 10 g of Perilla frutescens, 22 g of Platycodon grandiflorus, 9 g of star anise, 12 g of Aucklandia lappa, 8 g of Agastache rugosa, 11 g of Atractylodes lancea, 11 g of Atractylodes macrocephala, 11 g of green tangerine peel, 11 g of cloves, 5 g of Zanthoxylum bungeanum, and 15 g of tea leaves to obtain a Chinese herbal mixture, and the Chinese herbal mixture is added into a reaction vessel. Then, 500 g of deionized water is added into the reaction vessel to obtain a to-be-extracted Chinese herbal mixture. The to-be-extracted Chinese herbal mixture is performed with distillation extraction at 96° C. for 2.5 h to obtain the Chinese herbal extract.
(4) 12 g of rose hydrosol and 4 g of peppermint hydrosol are added into 40 g of Chinese herbal extract to obtain a stock solution. The stock solution is diluted 20 times to obtain a diluted stock solution, then 4 g of borneol is added into the diluted stock solution, stirred thoroughly and filtered to obtain a filtrate. 3 g of chelating platinum ions and 9 g of composite antibacterial agent are added into the filtrate, stirred evenly, so as to obtain the Chinese herbal feminine care solution.

### Embodiment 3

A preparation method of a Chinese herbal feminine care solution includes steps as follows.
(1) 10 g of nano-silica is dispersed into 50 g of 75wt % ethanol aqueous solution to obtain a first dispersed solution, then 8 g of 20wt % ammonia water and 3 g of 3-mercaptopropyltrimethoxysilane are added into the first dispersed solution to obtain a first mixed solution. The first mixed solution is stirred and reacted in a N₂ atmosphere at 20°° C. for 5 h to obtain a first product. The first product is centrifuged, washed and dried to obtain sulfhydrylated silica.
(2) 2 g of dodecfluoroheptyl methacrylate and 4 g of vinyl guanamine are added into 30 g of N,N-dimethylformamide, stirred evenly to obtain a second mixed solution, then 10 g of sulfhydrylated silica is dispersed into the second mixed solution by ultrasonic dispersion for 5 min to obtain a second dispersed solution, and 0.2 g of 2-ethoxy-1,2-diphenylethanone is added in the second dispersed solution. Under an irradiation of ultraviolet light with a main wavelength of 365 nm and an intensity of 100 mW/cm², a reaction is carried out at 30° C. for 30 min to obtain a second product. The second product is centrifuged, washed with isopropanol, dried, so as to obtain the composite antibacterial agent.
(3) 2 g of safflower, 10 g of peach seed, 10 g of Forsythia suspensa, 11 g of chrysanthemum, 10 g of Perilla frutescens, 22 g of Platycodon grandiflorus, 9 g of star anise, 12 g of Aucklandia lappa, 8 g of Agastache rugosa, 11 g of Atractylodes lancea, 11 g of Atractylodes macrocephala, 11 g of green tangerine peel, 11 g of cloves, 5 g of Zanthoxylum bungeanum, and 15 g of tea leaves to obtain a Chinese herbal mixture, and the Chinese herbal mixture is added into a reaction vessel. Then, 500 g of deionized water is added into the reaction vessel to obtain a to-be-extracted Chinese herbal mixture. The to-be-extracted Chinese herbal mixture is performed with distillation extraction at 96° C. for 2.5 h to obtain the Chinese herbal extract.
(4) 10 g of rose hydrosol and 3 g of peppermint hydrosol are added into 30 g of Chinese herbal extract to obtain a stock solution. The stock solution is diluted 20 times to obtain a diluted stock solution, then 3 g of borneol is added into the diluted stock solution, stirred thoroughly and filtered to obtain a filtrate. 1 g of chelating platinum ions and 6 g of composite antibacterial agent are added into the filtrate, stirred evenly, so as to obtain the Chinese herbal feminine care solution.

### Comparative Embodiment 1

A preparation method of a Chinese herbal feminine care solution includes steps as follows.
(1) 10 g of nano-silica is dispersed into 70 g of 75wt % ethanol aqueous solution to obtain a first dispersed solution, then 17 g of 20wt % ammonia water and 6 g of 3-mercaptopropyltrimethoxysilane are added into the first dispersed solution to obtain a first mixed solution. The first mixed solution is stirred and reacted in a N₂ atmosphere at 35° C. for 8 h to obtain a first product. The first product is centrifuged, washed and dried to obtain sulfhydrylated silica.
(2) 5 g of dodecfluoroheptyl methacrylate is added into 50 g of N,N-dimethylformamide, stirred evenly to obtain a second mixed solution, then 10 g of sulfhydrylated silica is dispersed into the second mixed solution by ultrasonic dispersion for 15 min to obtain a second dispersed solution, and 0.2 g of 2-ethoxy-1,2-diphenylethanone is added in the second dispersed solution. Under an irradiation of ultraviolet light with a main wavelength of 365 nm and an intensity of 100 mW/cm², a reaction is carried out at 40° C. for 60 min to obtain a second product. The second product is centrifuged, washed with isopropanol, dried, so as to obtain an antibacterial agent.
(3) 2 g of safflower, 10 g of peach seed, 10 g of Forsythia suspensa, 11 g of chrysanthemum, 10 g of Perilla frutescens, 22 g of Platycodon grandiflorus, 9 g of star anise, 12 g of Aucklandia lappa, 8 g of Agastache rugosa, 11 g of Atractylodes lancea, 11 g of Atractylodes macrocephala, 11 g of green tangerine peel, 11 g of cloves, 5 g of Zanthoxylum bungeanum, and 15 g of tea leaves to obtain a Chinese herbal mixture, and the Chinese herbal mixture is added into a reaction vessel. Then, 500 g of deionized water is added into the reaction vessel to obtain a to-be-extracted Chinese herbal mixture. The to-be-extracted Chinese herbal mixture is performed with distillation extraction at 96° C. for 2.5 h to obtain the Chinese herbal extract.
(4) 15 g of rose hydrosol and 5 g of peppermint hydrosol are added into 45 g of Chinese herbal extract to obtain a stock solution. The stock solution is diluted 20 times to obtain a diluted stock solution, then 5 g of borneol is added into the diluted stock solution, stirred thoroughly and filtered to obtain a filtrate. 5 g of chelating platinum ions and 12 g of antibacterial agent are added into the filtrate, stirred evenly, so as to obtain the Chinese herbal feminine care solution.

### Comparative embodiment 2

A preparation method of a Chinese herbal feminine care solution includes steps as follows.
(1) 10 g of nano-silica is dispersed into 70 g of 75wt % ethanol aqueous solution to obtain a first dispersed solution, then 17 g of 20wt % ammonia water and 6 g of 3-mercaptopropyltrimethoxysilane are added into the first dispersed solution to obtain a first mixed solution. The first mixed solution is stirred and reacted in a N₂ atmosphere at 35° C. for 8 h to obtain a first product. The first product is centrifuged, washed and dried to obtain sulfhydrylated silica.
(2) 7g of vinyl guanamine is added into 50 g of N,N-dimethylformamide, stirred evenly to obtain a second mixed solution, then 10 g of sulfhydrylated silica is dispersed into the second mixed solution by ultrasonic dispersion for 15 min to obtain a second dispersed solution, and 0.2 g of 2-ethoxy-1,2-diphenylethanone is added in the second dispersed solution. Under an irradiation of ultraviolet light with a main wavelength of 365 nm and an intensity of 100 mW/cm², a reaction is carried out at 40° C. for 60 min to obtain a second product. The second product is centrifuged, washed with isopropanol, dried, so as to obtain an antibacterial agent.
(3) 2 g of safflower, 10 g of peach seed, 10 g of Forsythia suspensa, 11 g of chrysanthemum, 10 g of Perilla frutescens, 22 g of Platycodon grandiflorus, 9 g of star anise, 12 g of Aucklandia lappa, 8 g of Agastache rugosa, 11 g of Atractylodes lancea, 11 g of Atractylodes macrocephala, 11 g of green tangerine peel, 11 g of cloves, 5 g of Zanthoxylum bungeanum, and 15 g of tea leaves to obtain a Chinese herbal mixture, and the Chinese herbal mixture is added into a reaction vessel. Then, 500 g of deionized water is added into the reaction vessel to obtain a to-be-extracted Chinese herbal mixture. The to-be-extracted Chinese herbal mixture is performed with distillation extraction at 96° C. for 2.5 h to obtain a Chinese herbal extract.
(4) 15 g of rose hydrosol and 5 g of peppermint hydrosol are added into 45 g of Chinese herbal extract to obtain a stock solution. The stock solution is diluted 20 times to obtain a diluted stock solution, then 5 g of borneol is added into the diluted stock solution, stirred thoroughly and filtered to obtain a filtrate. 5 g of chelating platinum ions and 12 g of antibacterial agent are added into the filtrate, stirred evenly, so as to obtain the Chinese herbal feminine care solution.

### Comparative embodiment 3

A preparation method of a Chinese herbal feminine care solution includes steps as follows.
(1) 10 g of nano-silica is dispersed into 70 g of 75wt % ethanol aqueous solution to obtain a first dispersed solution, then 17 g of 20wt % ammonia water and 6 g of 3-mercaptopropyltrimethoxysilane are added into the first dispersed solution to obtain a first mixed solution. The first mixed solution is stirred and reacted in a N₂ atmosphere at 35° C. for 8 h to obtain a first product. The first product is centrifuged, washed and dried to obtain sulfhydrylated silica.
(2) 2 g of safflower, 10 g of peach seed, 10 g of Forsythia suspensa, 11 g of chrysanthemum, 10 g of Perilla frutescens, 22 g of Platycodon grandiflorus, 9 g of star anise, 12 g of Aucklandia lappa, 8 g of Agastache rugosa, 11 g of Atractylodes lancea, 11 g of Atractylodes macrocephala, 11 g of green tangerine peel, 11 g of cloves, 5 g of Zanthoxylum bungeanum, and 15 g of tea leaves to obtain a Chinese herbal mixture, and the Chinese herbal mixture is added into a reaction vessel. Then, 500 g of deionized water is added into the reaction vessel to obtain a to-be-extracted Chinese herbal mixture. The to-be-extracted Chinese herbal mixture is performed with distillation extraction at 96°° C. for 2.5 h to obtain a Chinese herbal extract.
(3) 15 g of rose hydrosol and 5 g of peppermint hydrosol are added into 45 g of Chinese herbal extract to obtain a stock solution. The stock solution is diluted 20 times to obtain a diluted stock solution, then 5 g of borneol is added into the diluted stock solution, stirred thoroughly and filtered to obtain a filtrate. 5 g of chelating platinum ions and 12 g of sulfhydrylated silica are added into the filtrate, stirred evenly, so as to obtain the Chinese herbal feminine care solution.

Common pathogenic bacteria of the female vulva, such as Escherichia coli, Staphylococcus aureus, Pseudomonas aeruginosa, and Bacillus subtilis, are selected as experimental strains, and are activated in a broth medium at 37° C. for 8 h with a shaker at rotation speed of 150 revolutions per minute (rpm) for later use. The care solutions prepared in the embodiments 1-3 and the comparative embodiments 1-3, which have been sterilized at 121° C. for 20 min, are respectively added to a sterile broth medium at a concentration of 0.05% weight/volume, abbreviated as w/v). Under sterile conditions, 1 milliliter (mL) of activated bacterial solution (with a bacterial concentration of 10⁹ colony-forming units per milliliter, abbreviated as CFU/mL) is taken and added to 49 mL of mediums prepared as described above, and then is cultured at 37° C. for 6 h with a shaker at the rotation speed of 150 rpm to obtain a sample. After that, according to GB 4789.2-2022 “National Food Safety Standard-Microbiological Examination of Food: Enumeration of Total Bacterial Colonies”, the sample after diluting is characterized by a plate count method. A bactericidal rate is calculated using following formula: Bactericidal rate (%)=(Control group bacterial count−Experimental group bacterial count)/Control group bacterial count×100, where bacterial counts are expressed in CFU/mL. The experiment results are shown in Table 1.

TABLE 1
Bactericidal rate (%) of care solutions prepared
by different embodiments against bacteria

| | Escherichia | Staphylococcus | Pseudomonas | Bacillus |
|---|---|---|---|---|
| | coli | aureus | aeruginosa | subtilis |
| Embodiment 1 | 99.99 | 99.86 | 99.97 | 92.39 |
| Embodiment 2 | 99.87 | 99.67 | 99.83 | 91.75 |
| Embodiment 3 | 99.81 | 99.54 | 99.80 | 91.43 |
| Comparative | 85.96 | 82.33 | 79.14 | 72.57 |
| embodiment 1 | | | | |
| Comparative | 91.43 | 89.71 | 88.29 | 84.62 |
| embodiment 2 | | | | |
| Comparative | 79.31 | 76.45 | 71.72 | 63.96 |
| embodiment 3 | | | | |

The above description is only illustrated embodiments of the disclosure, but the scope of protection of the disclosure is not limited thereto. Any equivalent substitution or change made by any person skilled in the art according to the technical solutions of the disclosure and the inventive concept thereof within the technical scope disclosed by the disclosure shall be included in the scope of protection of the disclosure.

What is claimed is:

1. A formula for a Chinese herbal feminine care solution, wherein the formula for the Chinese herbal feminine care solution is made from raw materials in parts by weight: 10-15 parts of rose hydrosol, 3-5 parts of peppermint hydrosol, 30-45 parts of a Chinese herbal extract, 3-5 parts of borneol, 1-5 parts of chelated platinum ions, and 6-12 parts of composite antibacterial agent; and
wherein a preparation method of the composite antibacterial agent comprises the following steps:
(1) dispersing nano-silica into an ethanol aqueous solution to obtain a first dispersed solution, then adding ammonia water and 3-mercaptopropyltrimethoxysilane into the first dispersed solution to obtain a first mixed solution, stirring the first mixed solution for a reaction to obtain a first product, performing processes of centrifuging, washing and drying sequentially on the first product to obtain sulfhydrylated silica; and
(2) adding dodecfluoroheptyl methacrylate and vinyl guanamine into N,N-dimethylformamide, stirring evenly to obtain a second mixed solution, dispersing the sulfhydrylated silica into the second mixed solution by ultrasonic dispersion to obtain a second dispersed solution, adding a photoinitiator into the second dispersed solution for a reaction under an irradiation of an ultraviolet light to obtain a second product, centrifuging the second product to obtain a centrifuged second product, washing the centrifuged second product with isopropanol to obtain a washed second product, and drying the washed second product to obtain the composite antibacterial agent.

2. The formula for the Chinese herbal feminine care solution as claimed in claim 1, wherein, in the step (1), a concentration of the ethanol aqueous solution is in a range of 50 weight percent (wt %) to 75wt %, and a concentration of the ammonia water is in a range of 15wt % to 25wt %.

3. The formula for the Chinese herbal feminine care solution as claimed in claim 1, wherein, in the step (1), a weight ratio of the nano-silica, the ethanol aqueous solution, the ammonia water and the 3-mercaptopropyltrimethoxylsilane is 10: (50-70): (8-17): (3-6).

4. The formula for the Chinese herbal feminine care solution as claimed in claim 1, wherein, in the step (1), conditions of the reaction comprise: 5 to 8 hours (h) of reaction tine, 20 to 35 Celsius degrees (° C.) of reaction temperature, and a nitrogen (N₂) atmosphere.

5. The formula for the Chinese herbal feminine care solution as claimed in claim 1, wherein, in the step (2), a weight ratio of the dodecfluoroheptadyl methacrylate, the vinyl guanamine, the N,N-dimethylformamide and the sulfhydrylated silica is (2-5): (4-7): (30-50): 10.

6. The formula for the Chinese herbal feminine care solution as claimed in claim 1, wherein, in the step (2), the photoinitiator is one or more selected from the group consisting of consisting of 2,2-dimethoxy-2-phenylacetophenone, 2-ethoxy-1,2-diphenylethanone, and 2-butoxy-1,2-diphenylethanone.

7. The formula for the Chinese herbal feminine care solution as claimed in claim 1, wherein, in the step (2), time of the ultrasonic dispersion is in a range of 5 to 15 minutes (min), a main wavelength of the ultraviolet light is in a range of 360 to 365 nanometers (nm), an intensity of the ultraviolet light is in a range of 50 to 100 milliwatts per square centimeter (mW/cm²), time of the irradiation is in a range of 30 to 60 min, and a temperature of the reaction is in a range of 30 to 40° C.

8. The formula for the Chinese herbal feminine care solution as claimed in claim 1, wherein a preparation method of the Chinese herbal extract comprises the following steps:
weighing, in parts by weight, 1 to 2 parts of safflower, 8 to 12 parts of peach seed, 8 to 12 parts of Forsythia suspensa, 8 to 13 parts of chrysanthemum, 7 to 13 parts of Perilla frutescens, 18 to 25 parts of Platycodon grandiflorus, 8 to 11 parts of star anise, 9 to 14 parts of Aucklandia lappa, 7 to 12 parts of Agastache rugosa, 10 to 12 parts of Atractylodes lancea, 10 to 12 parts of Atractylodes macrocephala, 9 to 13 parts of green tangerine peel, 10 to 12 parts of cloves, 4 to 6 parts of Zanthoxylum bungeanum, and 13 to 16 parts of tea leaves to obtain a Chinese herbal mixture, adding the Chinese herbal mixture into a reaction vessel, then adding 500 parts of deionized water into the reaction vessel to obtain a to-be-extracted Chinese herbal mixture, and performing distillation extraction on the to-be-extracted Chinese herbal mixture at a temperature in a range of 94-97° C. for 1.5-3 h to obtain the Chinese herbal extract.

9. A preparation method of a Chinese herbal feminine care solution, comprising:
adding, in parts by weight, 10-15 parts of rose hydrosol and 3-5 parts of peppermint hydrosol into 30-45 parts of Chinese herbal medicine extract to obtain a stock solution; diluting the stock solution to obtain a diluted stock solution; adding 3-5 parts of borneol, stirring the diluted stock solution with the borneol to obtain a stirred solution, and filtering the stirred solution to obtain a filtrate, adding 1-5 parts of chelated platinum ions and 6-12 parts of composite antibacterial agent into the filtrate to stir evenly, so as to obtain the Chinese herbal feminine care solution;
wherein the composite antibacterial agent is prepared by:
step (1) dispersing nano-silica into an ethanol aqueous solution to obtain a first dispersed solution, then adding ammonia water and 3-mercaptopropyltrimethoxysilane into the first dispersed solution to obtain a first mixed solution, stirring the first mixed solution for a reaction to obtain a first product, performing centrifuging, washing and drying sequentially on the first product to obtain sulfhydrylated silica; and
step (2) adding dodecfluoroheptyl methacrylate and vinyl guanamine into N,N-dimethylformamide, stirring evenly to obtain a second mixed solution, dispersing the sulfhydrylated silica into the second mixed solution by ultrasonic dispersion to obtain a second dispersed solution, adding a photoinitiator into the second dispersed solution for a reaction under an irradiation of an ultraviolet light to obtain a second product, centrifuging the second product to obtain a centrifuged second product, washing the centrifuged second product with isopropanol to obtain a washed second product, and drying the washed second product to obtain the composite antibacterial agent.

10. The preparation method as claimed in claim 9, wherein, in the step (1), a concentration of the ethanol aqueous solution is in a range of 50 weight percent (wt %) to 75wt %, and a concentration of the ammonia water is in a range of 15wt % to 25wt %.

11. The preparation method as claimed in claim 9, wherein, in the step (1), a weight ratio of the nano-silica, the ethanol aqueous solution, the ammonia water and the 3-mercaptopropyltrimethoxylsilane is 10: (50-70): (8-17): (3-6).

12. The preparation method as claimed in claim 9, wherein, in the step (1), conditions of the reaction comprise: 5 to 8 hours (h) of reaction tine, 20 to 35 Celsius degrees (° C.) of reaction temperature, and a nitrogen (N₂) atmosphere.

13. The preparation method as claimed in claim 9, wherein, in the step (2), a weight ratio of the dodecfluoroheptadyl methacrylate, the vinyl guanamine, the N,N-dimethylformamide and the sulfhydrylated silica is (2-5): (4-7): (30-50): 10.

14. The preparation method as claimed in claim 1, wherein, in the step (2), the photoinitiator is one or more selected from the group consisting of consisting of 2,2-dimethoxy-2-phenylacetophenone, 2-ethoxy-1,2-diphenylethanone, and 2-butoxy-1,2-diphenylethanone.

15. The preparation method as claimed in claim 1, wherein, in the step (2), time of the ultrasonic dispersion is in a range of 5 to 15 minutes (min), a main wavelength of the ultraviolet light is in a range of 360 to 365 nanometers (nm), an intensity of the ultraviolet light is in a range of 50 to 100 milliwatts per square centimeter (mW/cm²), time of the irradiation is in a range of 30 to 60 min, and a temperature of the reaction is in a range of 30 to 40° C.

16. The preparation method as claimed in claim 1, wherein the Chinese herbal extract is prepared by the following steps:
weighing, in parts by weight, 1 to 2 parts of safflower, 8 to 12 parts of peach seed, 8 to 12 parts of Forsythia suspensa, 8 to 13 parts of chrysanthemum, 7 to 13 parts of Perilla frutescens, 18 to 25 parts of Platycodon grandiflorus, 8 to 11 parts of star anise, 9 to 14 parts of Aucklandia lappa, 7 to 12 parts of Agastache rugosa, 10 to 12 parts of Atractylodes lancea, 10 to 12 parts of Atractylodes macrocephala, 9 to 13 parts of green tangerine peel, 10 to 12 parts of cloves, 4 to 6 parts of Zanthoxylum bungeanum, and 13 to 16 parts of tea leaves to obtain a Chinese herbal mixture, adding the Chinese herbal mixture into a reaction vessel, then adding 500 parts of deionized water into the reaction vessel to obtain a to-be-extracted Chinese herbal mixture, and performing distillation extraction on the to-be-extracted Chinese herbal mixture at a temperature in a range of 94-97° C. for 1.5-3 h to obtain the Chinese herbal extract.

17. The preparation method as claimed in claim 9, wherein a dilution factor of the stock solution is in a range of 10-20 times.

18. A Chinese herbal feminine care solution, made from raw materials: rose hydrosol, peppermint hydrosol, a Chinese herbal extract, borneol, chelated platinum ions, and a composite antibacterial agent.

19. The Chinese herbal feminine care solution as claimed in claim 18, wherein the composite antibacterial agent is prepared by:
(1) dispersing 10 grams (g) nano-silica into 70 g of 75wt % an ethanol aqueous solution to obtain a first dispersed solution, then adding 17 g of 20wt % ammonia water and 6 g of 3-mercaptopropyltrimethoxysilane into the first dispersed solution to obtain a first mixed solution, stirring the first mixed solution for a reaction in a N₂ atmosphere at 35° C. for 8 h to obtain a first product, performing centrifuging, washing and drying sequentially on the first product to obtain sulfhydrylated silica; and
(2) adding 5 g of dodecfluoroheptyl methacrylate and 7 g of vinyl guanamine into 50 g of N,N-dimethylformamide, stirring evenly to obtain a second mixed solution, dispersing 10 g of the sulfhydrylated silica into the second mixed solution by ultrasonic dispersion for 15 min to obtain a second dispersed solution, adding 0.2 g of a photoinitiator into the second dispersed solution for a reaction at 40°° C. for 60 min under an irradiation of an ultraviolet light with a main wavelength of 365 nm and an intensity of 100 mW/cm² to obtain a second product, centrifuging the second product to obtain a centrifuged second product, washing the centrifuged second product with isopropanol to obtain a washed second product, and drying the washed second product to obtain the composite antibacterial agent.

20. The Chinese herbal feminine care solution as claimed in claim 19, wherein the Chinese herbal extract is prepared by the following steps:
weighing 2 g of safflower, 10 g of peach seed, 10 g of Forsythia suspensa, 11 g of chrysanthemum, 10 g of Perilla frutescens, 22 g of Platycodon grandiflorus, 9 g of star anise, 12 g of Aucklandia lappa, 8 g of Agastache rugosa, 11 g of Atractylodes lancea, 11 g of Atractylodes macrocephala, 11 g of green tangerine peel, 11 g of cloves, 5 g of Zanthoxylum bungeanum, and 15 g of tea leaves to obtain a Chinese herbal mixture, adding the Chinese herbal mixture into a reaction vessel, then adding 500 g of deionized water into the reaction vessel to obtain a to-be-extracted Chinese herbal mixture, and performing distillation extraction on the to-be-extracted Chinese herbal mixture at a temperature of 96° C. for 2.5 h to obtain the Chinese herbal extract.
